(19) United States
(12) Patent Application Publication
(10) Pub. No.: US 2009/0192525 A1
(43) Pub. Date: Jul. 30, 2009

(54) TAPE-BASED SKIN TREATMENT - A SAFE AND QUICK ALTERNATIVE TO SKIN RENEWAL

(76) Inventor: YuYing Tang, Hadley, MA (US)

Correspondence Address: MCCORMICK, PAULDING & HUBER LLP, CITY PLACE II, 185 ASYLUM STREET, HARTFORD, CT, 06103, US

(21) Appl. No.: 12/360,408

(22) Filed: Jan. 27, 2009

Related U.S. Application Data
(60) Provisional application No. 61/023,956, filed on Jan. 28, 2008.

(51) Int. Cl. A61B 17/50; B32B 27/30; C09J 7/02

(52) U.S. Cl. 606/131; 428/352

(57) ABSTRACT

A skin renewal sheet for exfoliating skin includes a flexible backing layer, a skin-friendly pressure-sensitive adhesive layer disposed on one side of the backing layer and a release layer disposed on the skin friendly pressure sensitive adhesive layer wherein the pressure sensitive adhesive layer has a 180° peel force of from about 0.5 lbs/in. to about 5.0 lbs/in.

### CROSS-REFERENCE TO RELATED APPLICATION

This application claims priority to U.S. Provisional Application No. 61/023,956, filed on Jan. 28, 2008, titled “TAPE-BASED SKIN TREATMENT—A SAFE AND QUICK ALTERNATIVE TO SKIN RENEWAL,” which is hereby incorporated by reference in its entirety.

### FIELD OF INVENTION

This invention describes a skin-friendly tape sheet for removing dead skin cells quickly and safely, providing a safer alternative to chemical peels and mechanical dermabrasion. This skin-friendly tape sheet utilizes a clean pressure-sensitive adhesive on a flexible backing, and the adhesive force of the tape to skin is controlled so it removes dead skin cells on the surface of the skin without pulling human hairs. The advantages of this skin tape are its applicability to all types of skins, its fast action, and convenience.

### BACKGROUND OF THE INVENTION

As we age, our skin gradually changes from a smooth and radiant young skin to a dull, rough, and wrinkled complexion. This change is partially caused by the slow down in the skin cell renewal cycle. Skin cells are generated in the bottom layer of epidermis, or stratum bassale, through cell division. The new cell moves through layers of the skin while changing its shape and chemical composition. It eventually moves to the top of the epidermis, stratum corneum dies and sheds off. As the skin renewal rate decreases, the dead skin cells do not shed as easily, and this leads to a rough and dull looking in the skin's texture.

Skin exfoliation, a way to remove the oldest skin cells that cling to the skin's outmost surface, helps increase the cell renewal rate of aged skin. Exfoliation not only leaves the skin smoother and fresher, it also makes it easier for skin care products such as antioxidants to penetrate through the epidermis layer into the underlying dermis layer. Two main types of skin exfoliation methods have been used so far: chemical and mechanical methods. Chemical exfoliation utilizes a weak acid such as alphahydroxy acids, betahydroxy acids, retinoic acids, or their esters, to burn away the dead cells and to stimulate skin cell proliferation. Mechanical exfoliation utilizes an abrasive to physically rub off the dead skin cells. These two approaches are sometimes combined, by incorporating skin exfoliation acids in a microdermabrasion cream, along with the inorganic or organic abrasive powders.

Two major drawbacks exist with the above exfoliation methods. First, people who have sensitive skin are excluded from these treatments since their skin cannot withstand the harshness of these methods without blistering, stinging, and other inflammatory symptoms. Second, even with non-sensitive skin types, it is difficult to determine the right level of treatment. Over exfoliation, where the skin cells in the dermis layers are removed, is common with the existing methods. Most people end up going to beauty salons and spas in order to make sure that the proper level of treatment is applied, making it a costly and time-consuming procedure; and the end results are still questionable since a high percentage people break out after a facial treatment.

Tape striping, where a flexible tape having a pressure-sensitive adhesive was pressed against a skin surface and pulled off to remove an outer layer of skin, was sometimes used to disrupt a skin water barrier for subsequent treatment [U.S. Pat. No. 5,720,963, U.S. Pat. No. 6,645,184]. In U.S. Pat. Nos. 6,290,659 and 6,563,012, John Hill described a skin exfoliation kit, which contains a plurality of flexible transparent sheets with an adhesive, a rod handle to be attached to an edge of the adhesive, and a transparent comparison sheet. Repeated tape striping was performed with the adhesive film until desired appearance was achieved.

Tape striping can cause skin irritation by pulling some of the hair off your skin, and by bringing irritating chemicals in contact with your skin. The irritating chemicals include residual monomers and initiators in the adhesive. These drawbacks might be the reasons no striping tapes are available on the consumer market yet.

With the forgoing problems and concerns in mind, it is the general object of the present invention to provide a solution for the above mentioned skin sensitivity and safety issues. A safe and easy skin exfoliation approach is described below.

### SUMMARY OF THE INVENTION

It is an object of the present invention to provide for safe and easy skin exfoliation.

It is another object of the present invention to provide for safe and easy skin exfoliation utilizing a sheet with skin-friendly adhesives.

It is yet another object of the present invention to provide for safe and easy skin exfoliation utilizing a sheet having several layers.

It is yet another object of the present invention to provide for safe and easy skin exfoliation utilizing a sheet having a predetermined peel force.

These and other objectives of the present invention, and their preferred embodiments, shall become clear by consideration of the specification, claims and drawings taken as a whole.

### BRIEF DESCRIPTION OF THE DRAWINGS

FIG. 1 is a partial perspective view of the skin-renewal sheet of the present invention.

FIG. 2 is a plan view of the skin-renewal sheet of the present invention in the form of a facemask.

### DETAILED DESCRIPTION OF THE PREFERRED EMBODIMENT

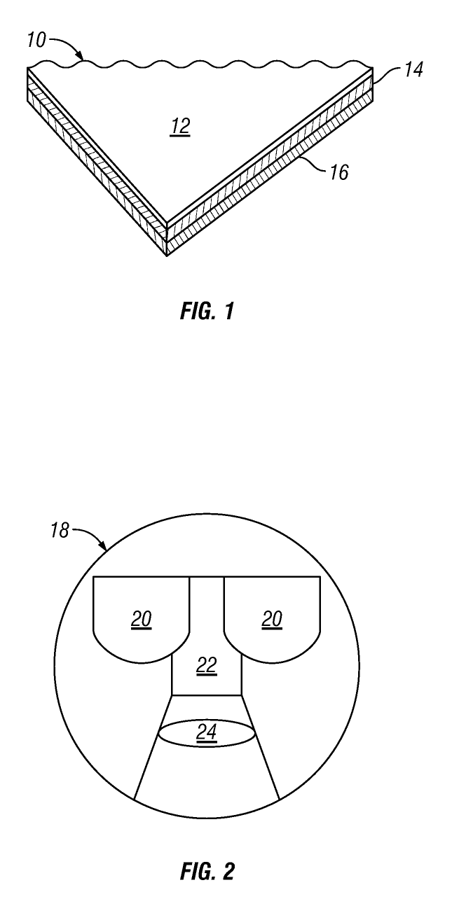

A skin-renewal sheet or tape utilizing clean and skin-friendly adhesives is employed to remove the dead skin cells on the stratum corneum layer. This sheet or tape is brought into contact with a person's skin, and then removed slowly after the desired amount of contact time. The contact time can be ranged from a few seconds to a few minutes, i.e. from about 5 seconds to about 5 minutes, depending on the user's preference. The removal of the sheet or tape takes away layers of dead skin cells. It also takes any dirt it comes into contact with. Over exfoliation is not possible with this method, since the detachment of the dead skin cells occurs by cohesive breakdown of the stratum corneum layer, which can occur for only the more weakly bound outer cells of this layer. With reference to FIG. 1, the sheet or tape 10 is comprised of 3 layers, i.e., a flexible backing layer 12, a skin-friendly adhesive layer 14 and a release layer 16.

The adhesive used for the flexible sheet or tape needs to be skin-friendly. It is preferred that the adhesive has passed the following tests: cytotoxicity-ISO10993-5, skin sensitization-ISO 10993-10, and irritation-ISO10993-10. It is also preferred that the dry film components comply with the compositional requirements of the FDA indirect Food Additive Regulations: 21 CFR 175.105 “Adhesives”.

The peel force of the adhesive needs to be in the range of 0.5-5 lb/in, and more preferable in the range of 0.8-3 lb/in (PSTC-101: International Standard for Peel Adhesion of Pressure Sensitive Tape). When the peel force is too low, the tape can be removed from the skin cleanly without detaching a layer or layers of dead skin cells, rendering the treatment ineffective. When the peel force is too high, the removal of the tape will be painful due to the tape's pull on skin hairs.

The thickness of the pressure sensitive adhesive is preferred to be in the range of 0.5 mil to 10 mil, more preferably in the range of 1 mil to 6 mil. If the thickness of the adhesive is below 0.5 mil, the adhesive does not have close contact with the micro-rough skin and renders it less effective. If the thickness of the adhesive is greater than 10 mil, the whole construction becomes too soft and less convenient for handling.

The backing of the sheet or tape can be any flexible carrier such as fabrics, polyurethane films, ethylene-vinyl acetate copolymer films, ethylene-acrylic acid copolymer films, polyester films, polyolefin films, nylons, vinyl films, nonwoven cloth, polyethylene foam sheets, polyurethane foam sheets, vinyl foam sheets, paper, any other clear or opaque flexible carriers, laminates of the above films or sheets. The adhesive needs to anchor well to the tape backing, to ensure that the adhesive will stay with its carrier during use.

Examples of adhesives that can be used for this application include, but are not limited to, the following: pressure sensitive acrylic adhesives, polyesters, polyurethanes, synthetic rubbers, silicone copolymers, polyisobutylenes, and ethylene vinyl acetates. Blends of more than one adhesive can be used to obtain the desired performance. These adhesives can be cast from organic solvent solution or aqueous emulsion. They can also be made by a hot-melt extrusion method or by radiation cure such as ultraviolet radiation cure or electron bean cure.

Other skin-friendly additives can be present in the adhesive layer in a total amount of less than 40 percent of the total adhesive layer, preferable in a total amount of less than 25 percent of the total layer. Examples of these additives include emollients such as polyethelene glycol, propropylene glycol, glycerine, and olive oil. Inorganic and organic fillers such as zinc oxide and colorants can also be present. The purpose of skin emollients in the adhesive is to further reduce discomfort during tape peel off.

Use of skin renewal sheet of the present invention involves the exfoliation of a portion of epidermis from a selected portion of the human body, applying the skin renewal sheet having a size and shape adapted to fit the selected portion, leaving the skin renewal sheet with the skin-friendly pressure-sensitive adhesive in contact with the epidermis for a period of time and removing the skin renewal sheet from the selected portion of the human body.

In accordance with the above, the skin renewal sheet may be supplied in a shape of facial mask such as shown at 18 in FIG. 2, in strips, in shapes of other body parts where exfoliation is desired, or in a combination of the above formats. When it is supplied as a facial mask 18, the sheet or tape can be a whole facial mask with cut out eyes 20, nose 22, and mouth region 24. It can also be a multi-parts facial mask, where different parts fitting different areas of the face can be peeled off and used separately.

The skin renewal sheet is further illustrated by the examples below.

### EXAMPLES

### Example 1

Gelva GMS 2499, a skin-friendly acrylic pressure sensitive adhesive, was cast on 2 mil Duraflex PS8000, a clear polyurethane film, and the solvent was allowed to be dried off to obtain a dried adhesive thickness of 1 mil. The dried adhesive was then covered with a 76# siliconized release paper Grade 32288 S80# BL SCK 1100/PE from Loparex. The 1800 peel force was then tested according to PSTC-101 and the peel force was 2.7 lb/inch after 20 minutes dwell time.

The sheet was then cut into 1″×2″ strips and placed on washed and dried skin of the forehead and cheeks and left for one minute before removing it slowly at 180° angle. The removed tape was observed under a microscope and the coverage of the dead skin cells on the tape was approximately 84% area. No uncomfortable feeling or hair pulling was felt on the skin when the tape was being removed. The treated skin felt nice and smooth.

### Example 2

A skin-friendly rubber pressure sensitive adhesive, DERMA-TAK 36-614A, was cast on a 2 mil, clear, low density polyethylene film and the solvent was allowed to dry off completely to obtain a dried adhesive thickness of 2 mil. The polyethylene film was corona-treated before the adhesive was cast on it to obtain good adhesion between the adhesive and the film. The dried adhesive was covered with a siliconized release paper and cut into the desired size for use. The 180° peel force was then tested according to PSTC-101 and the peel force was 1.4 lb/inch after 20 minutes dwell time.

The sheet was cut into a 1″×2″ strips and placed on the washed and dried skin of the forehead and cheeks and left for one minute before removing it slowly at 180° angle. The removed tape was observed under a microscope and the coverage of the dead skin cells of the tape was approximately 81% of the tape surface. No uncomfortable feeling or hair pulling was felt on the skin when the tape was removed. The treated skin felt nice and smooth.

### Example 3

A skin-friendly silicone adhesive, BIO-PSA 7-4602, is cast on a 1 mil polyester film and this 1 mil polyester film is supported on a carrier sheet such as a release paper. The silicone adhesive is allowed to dry out and obtain a dried thickness of 1.5 mil and the 180° peel force of this adhesive is 2.7 lb/in according to the supplier, Dow Corning. This construction is then be covered by a release paper, and cut into either strips or into a shape of a facial mask as illustrated in FIG. 2. Facial skin exfoliation is then performed by removing the pieces one by one and positioning it on the corresponding part on the face followed by removal.

### Example 4

A skin-friendly hot melt silicone adhesive, BIO-PSA 7-4560, is cast with a hot slot-die onto a release carrier sheet to obtain a 2 mil adhesive. The 180° peel force of this 2 mil adhesive is 2.5 lb/in according to the supplier, Dow Corning. This carrier sheet is then laminated to a medical grade flexible vinyl film, such as those available from Teknor Apex Company. The adhesive bonds to the vinyl film during lamination, and the release carrier sheet stays until peeled away by end uses. The tape is supplied in the shape of strips and/or masks.

### Example 5

A skin-friendly solution acrylic adhesive, Gelva GMS 737, is mixed with an extra virgin cold pressed olive oil, in the solid ratio of 95:5. The mixture solution is cast on a silicone release paper, such as Loparex Grade 32288 by Mondi Coatings, and dried completely to obtain a 4 mil dried adhesive film. The dried adhesive side is then brought into contact with a nonwoven carrier such as DuPont Sotesse Medical Fabric, a spunlaced polyester fabric. The adhesive transfers to the fabric during lamination, and the release paper stays until peeled away by end users. The tape is supplied in the shape of strips and/or masks.

### Example 6

A skin-friendly polyisobutylene rubber pressure sensitive adhesive, DURO-TAK 87-608A, is cast on a silicone release paper, such as Loparex Grade 32288 by Mondi Coatings, and dried completely to obtain a 1 mil dried adhesive film. The 180° peel force of this adhesive is 2.9 lb/in according to the supplier, National Starch and Chemical. The dried adhesive is then brought into contact with a clear 2.3 mil ethylene vinylacetate (EVA) film supplied by Bloomer Plastics. The adhesive transfers to the EVA film during lamination, and the release paper stays until peeled away by end users. The tape is supplied in the shape of strips and/or masks.

While the invention has been described with reference to the preferred embodiments, it will be understood by those skilled in the art that various obvious changes may be made, and equivalents may be substituted for elements thereof, without departing from the essential scope of the present invention. Therefore, it is intended that the invention not be limited to the particular embodiments disclosed, but that the invention includes all embodiments falling within the scope of the appended claims.

What is claimed is:

1. A skin renewal sheet for exfoliating skin, said sheet comprising a flexible backing layer, a skin-friendly pressure-sensitive adhesive layer disposed on one side of the backing layer and a release layer disposed on the skin friendly pressure sensitive adhesive layer wherein the pressure sensitive adhesive layer has a 180° peel force of from about 0.5 lbs/in. to about 5.0 lbs/in.

2. The skin renewal sheet of claim 1 wherein the pressure sensitive adhesive layer has a 180° peel force of from about 0.8 lbs/in. to about 3.0 lbs/in.

3. The skin renewal sheet of claim 1 wherein the pressure sensitive adhesive layer has a thickness of from about 0.5 mil to about 10 mil.

4. The skin renewal sheet of claim 3 wherein the pressure sensitive adhesive layer has a thickness of from about 1 mil to about 6 mil.

5. The skin renewal sheet of claim 1 wherein the skin-friendly adhesive layer contains additives selected from the group consisting of emollients, inorganic and organic fillers and colorants.

6. The skin renewal sheet of claim 1 wherein the pressure sensitive adhesive layer is a member of the group consisting of an acrylic adhesive, a polyester adhesive, a polyurethane adhesive, a synthetic rubber adhesive, a silicone copolymer adhesive, a polyisobutylene adhesive, an ethylene vinyl acetate adhesive and blends thereof.

7. The skin renewal sheet of claim 1 wherein the pressure sensitive adhesive layer is an acrylic adhesive.

8. The skin renewal sheet of claim 1 wherein sheet is in the form of a tape.

9. The skin renewal sheet of claim 1 wherein sheet is in the form of a facemask.

10. A method of exfoliating a portion of epidermis from a selected portion of the human body comprising applying a skin renewal sheet having a size and shape adapted to fit the selected portion wherein the skin renewal sheet comprises a flexible backing layer and a skin-friendly pressure-sensitive adhesive layer having a 180° peel force of from about 0.5 lbs/in. to about 5.0 lbs/in disposed on one side of the backing layer, leaving the skin renewal sheet with the skin-friendly pressure-sensitive adhesive in contact with the epidermis for about 5 seconds to about 5 minutes and removing the skin renewal sheet from the selected portion of the human body.

11. The method of claim 10 wherein the skin-friendly adhesive is an acrylic adhesive.
